(19) United States
(12) Patent Application Publication
(10) Pub. No.: US 2023/0286921 A1
(43) Pub. Date: Sep. 14, 2023

(54) Method for Manufacturing Compound

(71) Applicant: Bioradical Research Institute Corp., Yokohama-shi, Kanagawa (JP)

(72) Inventors: Keisuke Makino, Kyoto-shi, Kyoto (JP); Toshiyuki Arai, Kyoto-shi, Kyoto (JP); Masaichi Lee, Yokohama-shi, Kanagawa (JP)

(73) Assignee: Bioradical Research Institute Corp., Yokohama-shi, Kanagawa (JP)

(21) Appl. No.: 18/017,961

(22) PCT Filed: Jul. 28, 2021
(86) PCT No.: PCT/JP2021/027915

§ 371 Date: Jan. 25, 2023

(30) Foreign Application Priority Data
Jul. 29, 2020 (JP) 2020-128216

(51) Int. Cl. C07D 231/20

(52) U.S. Cl. CPC C07D 231/20

(57) ABSTRACT

The present invention provides a method for manufacturing a compound, with which it is possible to improve the yield of the compound. The method for manufacturing a compound according to the present invention includes a first step of reacting a compound represented by formula (1) shown below with a compound represented by formula (2) shown below to obtain a compound represented by formula (3) shown below:

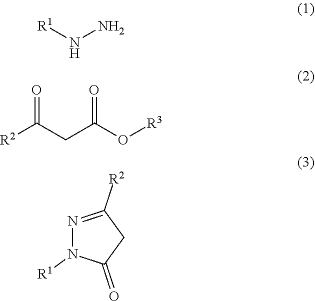

### TECHNICAL FIELD

The present invention relates to a method for manufacturing a compound and applications thereof.

### BACKGROUND ART

Reactive oxygen species are generated by oxygen metabolism that is essential for life activities. Also, because reactive oxygen species are highly reactive, excessive generation thereof induces tissue and cell damage, and thus it has been reported that reactive oxygen species are involved in various diseases. Accordingly, reactive oxygen species removing agents for removing the reactive oxygen species have been developed. However, most reactive oxygen species removing agents are unstable. For this reason, only edaravone (5-methyl-2-phenyl-2,4-dihydro-3H-pyrazol-3-one, product name: Radicut available from Mitsubishi Tanabe Pharma Corporation) is practically applied to clinical use as a reactive oxygen species removing agent (Non Patent Literature 1).

### CITATION LIST

### Non Patent Literature

Non Patent Literature 1: Piyanart Sommani, et. al., “Effects of Edaravone on Singlet Oxygen Released From Activated Human Neutrophils,” J. Pharmacol. Sci., 2007, vol. 103, pages 117 to 120

### SUMMARY OF INVENTION

### Technical Problem

Edaravone is an antioxidant that functions to protect the cerebral nerves by scavenging radicals, in particular, singlet oxygen generated when the blood flow resumes after an acute cerebral ischemic attack or cerebral infarction, and is a powerful radical scavenger. However, edaravone is unstable in an aqueous solution and easily oxidized. For this reason, it is required to be stored in a reducing aqueous solution.

The inventors of the present invention found that, when two substituted products, each obtained by substituting the phenyl group at the 2 position of edaravone by an alkyl group, are crosslinked using an alkenyl group, it is possible to obtain edaravone that is stable even in an aqueous solution while maintaining the antioxidation effect. The substituted products can also be prepared by reacting 3-methyl-3-3H-pyrazole-3-one (3-methyl-5-pyrazolone) with a bromoalkyl. However, the reaction for obtaining the substituted products is problematic in that the yield is not good.

To address this, it is an object of the present invention to provide a method for manufacturing a compound, with which it is possible to improve the yield of the compound.

### Solution to Problem

In order to achieve the object described above, a method for manufacturing a compound according to the present invention (hereinafter, also referred to as a “first manufacturing method”) includes a first step of reacting a compound represented by formula (1) shown below with a compound represented by formula (2) shown below to obtain a compound represented by formula (3) shown below:

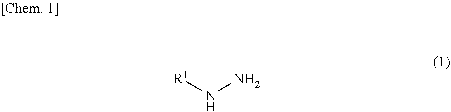

where R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent;

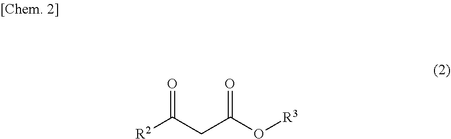

where R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R³ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; and

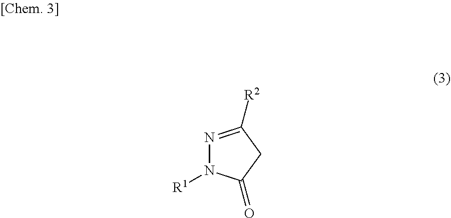

A method for manufacturing a compound according to the present invention (hereinafter, also referred to as a “second manufacturing method”) includes a first step of reacting a compound represented by formula (4) shown below with R¹—I to obtain a compound represented by formula (3) shown below, wherein R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent:

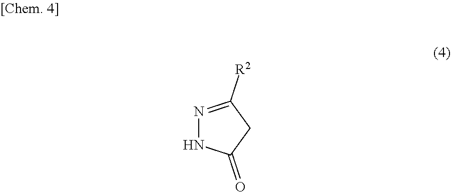

where R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; and

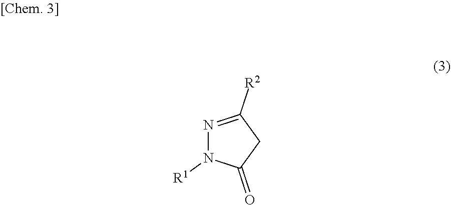

A method for manufacturing a compound according to the present invention (hereinafter, also referred to as a “third manufacturing method”) includes a first step of obtaining a compound represented by formula (3) shown below; and a second step of reacting the compound represented by formula (3) shown below with a compound represented by formula (5) shown below to obtain a compound represented by formula (6) shown below [ML1][ [symbol] 2],
wherein the first step is carried out using at least one of the first manufacturing method and the second manufacturing method of the present invention described above:

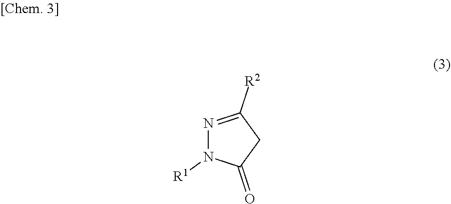

where R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent;

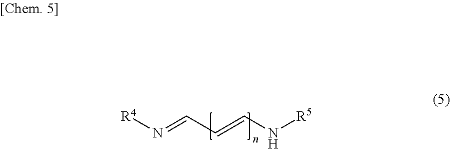

where R⁴ and R⁵ may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and n is an integer of 0 or more; and

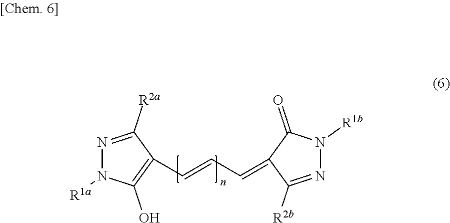

where R^{1a} and R^{1b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent,
R^{2a} and R^{2b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
n is an integer of 0 or more.

### Advantageous Effects of Invention

According to the present invention, it is possible to provide a method for manufacturing a compound, with which it is possible to improve the yield of the compound.

### BRIEF DESCRIPTION OF DRAWINGS

FIG. 1 shows graphs showing LC/MS results obtained in Example 1.

FIG. 2 shows graphs showing LC/MS results obtained in Example 1.

FIG. 3 shows a photographic image showing TLC results obtained in Example 1.

FIG. 4 shows graphs showing LC/MS results obtained in Example 1.

FIG. 5 shows graphs showing LC/MS results obtained in Example 1.

FIG. 6 shows a photographic image showing TLC results obtained in Example 1.

FIG. 7 shows graphs showing LC/MS results obtained in Example 1.

FIG. 8 shows a photographic image showing TLC results obtained in Example 1.

FIG. 9 shows graphs showing HPLC results obtained in Example 1.

### DESCRIPTION OF EMBODIMENTS

### First Manufacturing Method

As described above, a method for manufacturing a compound according to the present invention includes a first step (hereinafter, also referred to as “step 1A”) of reacting a compound represented by formula (1) shown below with a compound represented by formula (2) shown below to obtain a compound represented by formula (3) shown below.

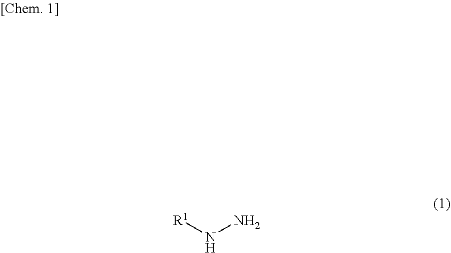

In formula (1) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

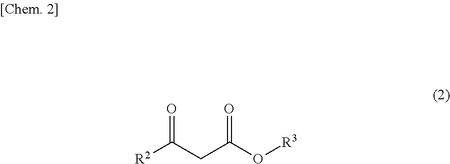

In formula (2) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.
R³ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

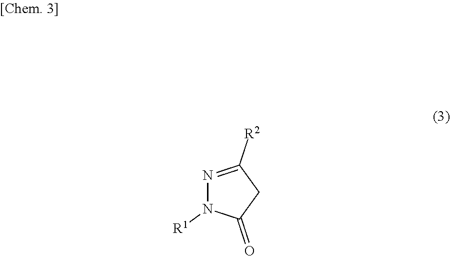

The compound represented by formula (1), (2), or (3) shown above may be, for example, a salt or an isomer thereof. Examples of the isomer include a tautomer and a stereoisomer. Examples of the tautomer and the stereoisomer include all theoretically possible tautomers and stereoisomers. Also, in the present invention, there is no particular limitation on the stereochemistry of each substituent. In the antioxidant of the present invention, the compound represented by formula (1), (2), or (3) shown above may be, for example, a hydrate or solvate of the compound represented by formula (1), (2), or (3) shown above or a salt thereof.

In the case where the compound represented by formula (1), (2), or (3) shown above has an asymmetric carbon atom, the compound represented by formula (1), (2), or (3) shown above may be present as, for example, a racemate, an R enantiomer or an S enantiomer of the racemate, or a mixture of the R and S enantiomers mixed at any ratio. The compound represented by formula (1), (2), or (3) shown above may have two or more asymmetric centers. In this case, the compound represented by formula (1), (2), or (3) shown above may contain a diastereoisomer and a mixture thereof. In the case where the compound represented by formula (1), (2), or (3) shown above has a double bond in the molecule, the compound represented by formula (1), (2), or (3) shown above may include the form of geometrical isomers such as cis and trans isomers.

Hereinafter, the substituents of the compounds will be described, and examples of each substituent will be given. In the description of each substituent, unless otherwise stated, specific examples of other substituents can be incorporated by reference.

In formula (1) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group or an aryl group, and more preferably represents an ethyl group or a phenyl group.

Examples of the halogen atom include a fluorine atom, a chlorine atom, a bromine atom, an iodine atom, and the like.

The alkyl group may be, for example, a linear, branched or cyclic saturated or unsaturated alkyl group having 1 to 20 or 1 to 10 carbon atoms. Specific examples of the alkyl group include a methyl group, an ethyl group, an n-propyl group, an i-propyl group, an n-butyl group, an i-butyl group, a t-butyl group, an n-pentyl group, an i-pentyl group, a t-pentyl group, an n-hexyl group, an i-hexyl group, a t-hexyl group, an n-heptyl group, an i-heptyl group, a t-heptyl group, an n-octyl group, an i-octyl group, a t-octyl group, an n-nonyl group, an i-nonyl group, a t-nonyl group, an n-decyl group, an i-decyl group, a t-decyl group, an n-undecyl group, an i-undecyl group, an n-dodecyl group, an i-dodecyl group, an n-tridecyl group, an i-tridecyl group, an n-tetradecyl group, an i-tetradecyl group, an n-pentadecyl group, an i-pentadecyl group, an n-hexadecyl group, an i-hexadecyl group, an n-heptadecyl group, an i-heptadecyl group, an n-octadecyl group, an i-octadecyl group, an n-nonadecyl group, an i-nonadecyl group, and the like. The alkyl group is preferably, for example, a linear saturated alkyl group having 1 to 6 carbon atoms, and more preferably a methyl group or an ethyl group.

In the alkoxy group (RO—), R represents an alkyl group, and the description of the alkyl group given above can be incorporated by reference.

In the hydroxyalkyl group (HOR—), R represents an alkyl group, and the description of the alkyl group given above can be incorporated by reference.

In the acyl group (RCO—), R represents an alkyl group, and the description of the alkyl group given above can be incorporated by reference.

The alkenyl group may be, for example, an alkenyl group obtained by forming one or a plurality of double bonds in any of the alkyl groups listed above, or the like. The alkenyl group may be, for example, an alkenyl group having 2 to 20 carbon atoms, and preferably 2 to 6 carbon atoms. Specific examples include a vinyl group, an allyl group, a 1-propenyl group, a 2-propenyl group, an isopropenyl group, a 1-butenyl group, a 2-butenyl group, a 3-butenyl group, a 2-methylallyl group, a 1-pentenyl group, a 2-pentenyl group, a 3-pentenyl group, a 4-pentenyl group, a 2-methyl-2-butenyl group, and the like.

The alkynyl group may be, for example, an alkynyl group obtained by forming one or a plurality of triple bonds in any of the alkyl groups listed above, or the like. The alkynyl group may be, for example, an alkenyl group having 2 to 20 carbon atoms, and preferably 2 to 6 carbon atoms. Specific examples include an etynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-3-butynyl group, and the like. The alkynyl group may further have, for example, one or a plurality of double bonds.

The aryl group optionally having a substituent may be an aryl group, or the aryl group may be substituted by a substituent. The aryl group optionally having a substituent may be, for example, an aryl group having 6 to 20 carbon atoms in total including carbon atoms in the substituent. Specific examples include a phenyl group, a tolyl group, a xylyl group, an alkyloxyphenyl group (for example, a methoxyphenyl group, an ethoxyphenyl group, or the like), a hydroxyphenyl group, a halogenophenyl group (for example, a fluorophenyl group, a chlorophenyl group, a bromophenyl group, or the like), an alkylphenyl group (for example, a methylphenyl group, an ethylphenyl group, a propylphenyl group, or the like), a cyanophenyl group, a propyloxyphenyl group, a 4-sulfophenyl group, and the like. The aryl group optionally having a substituent is preferably a phenyl group or a 4-sulfophenyl group.

In formula (2) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group, and more preferably represents a methyl group.

In formula (2) shown above, R³ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group, and more preferably represents an ethyl group.

There is no particular limitation on the combination of R¹, R², and R³, and they can be combined in any way. A specific example of the combination of R¹, R², and R³ is a combination where R represents an alkyl group or an aryl group, R² represents an alkyl group, and R³ represents an alkyl group, and the combination of R¹, R², and R³ is preferably a combination where R represents an ethyl group or a phenyl group, R² represents a methyl group, and R³ represents an ethyl group.

Next, the step of the first manufacturing method of the present invention will be described based on an example. In the first manufacturing method of the present invention, in the step TA, the compound represented by formula (1) shown above is reacted with the compound represented by formula (2) shown above to obtain the compound represented by formula (3) shown above. In the step TA, the compound represented by formula (3) shown above can be obtained through, for example, a reduction reaction between the compound represented by formula (1) shown above and the compound represented by formula (2) shown above. Specifically, in the step TA, as shown by a reaction formula (Chem. S1) given below, it is estimated that the amino group (—NH₂) and the amine (—NH—) in the compound represented by formula (1) reduce the carbonyl group in the compound represented by formula (2) to obtain the compound represented by formula (3).

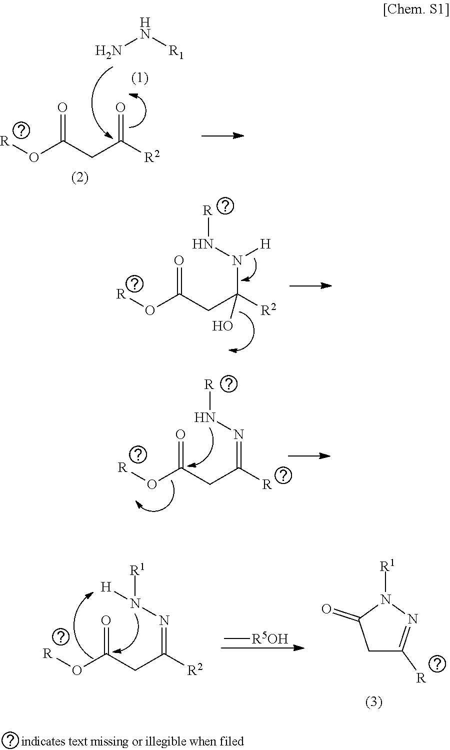

In the step 1A, the molar ratio (C1:C2) of the compound represented by formula (1) shown above (C1) and the compound represented by formula (2) shown above (C2) is, for example, 1:0.1 to 1:10, 1:0.25 to 1:5, or 1:0.5 to 1:1.25.

The step 1A may be carried out in the presence of a base, more specifically in the presence of a basic substance. Examples of the base include basic inorganic salts such as calcium carbonate, potassium carbonate, and cesium carbonate; metal hydrides such as sodium hydride and calcium hydride; amines such as triethylamine (TEA) and N,N-diisopropylethylamine (DIEA); and the like.

As the reaction solvent used in the step 1A, it is possible to use at least one solvent selected from, for example, N,N-dimethylformamide (DMF), acetonitrile, 1,4-dioxane, acetone, ethanol, chloroform, dioxane, xylene, diethyl ether, tetrahydrofuran (THF), dichloromethane (CH₂Cl₂), toluene, and the like. As the reaction solvent, these solvents may be used alone or in a combination of two or more, or a solvent mixture composed of two or more solvents selected from those listed above may be used. In the case where the step 1A is carried out in the presence of a base, and the base can be used as a reaction solvent, the base may be used as the reaction solvent in the step 1A. The base that can be used as the reaction solvent may be, for example, an amine such as TEA or DIEA. The reaction solvent is preferably, for example, DMF, acetonitrile, ethanol, an amine, or any of the solvent mixtures thereof because side reactions and the generation of isomers can be suppressed, and the yield of the compound represented by formula (3) shown above can be improved. The amount of the reaction solvent may be, for example, 0.5 to 1000 ml or 50 to 1000 ml relative to 1 mol of the compound represented by formula (1) shown above.

The reaction temperature in the step 1A can be adjusted as appropriate according to the compound represented by formula (1) or (2) shown above, the reaction solvent, and the like. As a specific example, the reaction temperature may be, for example, 0 to 40° C., 0 to 35° C., or the like.

In the first manufacturing method of the present invention, the compound represented by formula (3) obtained in the step 1A may be purified. In this case, in the first manufacturing method of the present invention, for example, the reaction product obtained in the step 1A may be subjected to processing such as recrystallization, concentration, neutralization, extraction, and/or drying.

According to the first manufacturing method of the present invention, the yield of the compound represented by formula (3) shown above may be improved.

### Second Manufacturing Method

As described above, a method for manufacturing a compound according to the present invention includes a first step (hereinafter, also referred to as “step 1B”) of reacting a compound represented by formula (4) shown below with R¹—I to obtain a compound represented by formula (3) shown below, wherein R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

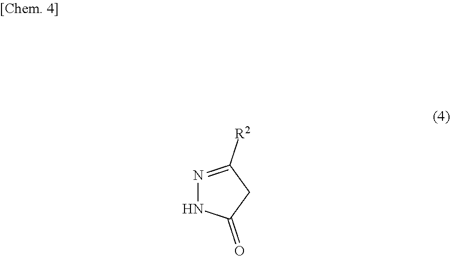

In formula (4) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

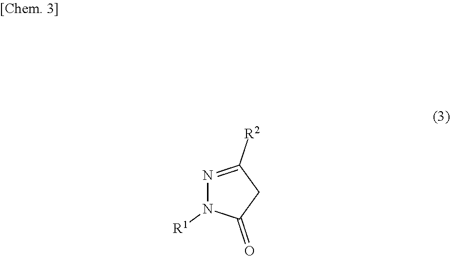

In the second manufacturing method of the present invention, the description of the first manufacturing method of the present invention given above can be incorporated by reference.

The compound represented by formula (3) or (4) shown above may be, for example, a salt or an isomer thereof. Examples of the isomer include a tautomer and a stereoisomer. Examples of the tautomer and the stereoisomer include all theoretically possible tautomers and stereoisomers. Also, in the present invention, there is no particular limitation on the stereochemistry of each substituent. In the antioxidant of the present invention, the compound represented by formula (3) or (4) shown above may be, for example, a hydrate or solvate of the compound represented by formula (3) or (4) shown above or a salt thereof.

In the case where the compound represented by formula (3) or (4) shown above has an asymmetric carbon atom, the compound represented by formula (3) or (4) shown above may be present as, for example, a racemate, an R enantiomer or an S enantiomer of the racemate, or a mixture of the R and S enantiomers mixed at any ratio. The compound represented by formula (3) or (4) shown above may have two or more asymmetric centers. In this case, the compound represented by formula (3) or (4) shown above may contain a diastereoisomer and a mixture thereof. In the case where the compound represented by formula (3) or (4) shown above has a double bond in the molecule, the compound represented by formula (3) or (4) shown above may include the form of geometrical isomers such as cis and trans isomers.

Hereinafter, the substituents of the compounds will be described, and examples of each substituent will be given. In the description of each substituent, unless otherwise stated, specific examples of other substituents can be incorporated by reference.

In R¹—I (iodine), R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group (alkyl iodide) or an aryl group, and more preferably represents an ethyl group (ethyl iodide) or a phenyl group (iodobenzene). It is estimated that, by using an iodide in the second manufacturing method of the present invention, the introduction efficiency of the R¹ group into the nitrogen atom at the 1 position of the pyrazoline ring is improved as compared with the case where a bromoalkyl group is used, and thus the yield is improved.

In formula (4) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group, and more preferably represents a methyl group.

There is no particular limitation on the combination of R¹ and R², and they can be combined in any way. A specific example of the combination of R¹ and R² is a combination where R¹ represents an alkyl group or an aryl group and R² represents an alkyl group, and the combination of R¹ and R² is preferably a combination where R¹ represents an ethyl group or a phenyl group, and R² represents a methyl group.

Next, the step of the second manufacturing method of the present invention will be described based on an example. In the second manufacturing method of the present invention, in the step 1B, the compound represented by formula (4) shown above is reacted with R¹—I to obtain the compound represented by formula (3) shown above. In the step 1B, the compound represented by formula (3) shown above can be obtained through, for example, a nucleophilic substitution reaction between the compound represented by formula (4) shown above and R¹—I. Specifically, in the step 1B, for example, the amine (—NH—) in the compound represented by formula (4) shown above functions as a nucleophilic species and acts on R¹—I, as a result of which, the compound represented by formula (3) shown above can be obtained.

In the step 1B, the molar ratio (C4:CI) of the compound represented by formula (4) shown above (C4) and R¹—I (CI) is, for example, 1:0.1 to 1:100, 1:0.5 to 1:50, or 1:1 to 1:25.

The step 1B is preferably carried out, for example, in the absence of a base, more specifically, in a reaction system that does not contain a basic substance because side reactions and the generation of isomers can be suppressed, and the yield of the compound represented by formula (3) shown above can be improved. The description of the base given above can be incorporated by reference.

As the reaction solvent used in the step 1B, it is possible to use at least one solvent selected from, for example, N,N-dimethylformamide (DMF), acetonitrile, 1,4-dioxane, acetone, ethanol, chloroform, dioxane, xylene, diethyl ether, tetrahydrofuran (THF), dichloromethane (CH₂Cl₂), toluene, and the like. As the reaction solvent, these solvents may be used alone or in a combination of two or more, or a solvent mixture composed of two or more solvents selected from those listed above may be used. The reaction solvent is preferably, for example, DMF, acetonitrile, or any of the solvent mixtures thereof because side reactions and the generation of isomers can be suppressed, and the yield of the compound represented by formula (3) shown above can be improved. The amount of the reaction solvent may be, for example, 0.5 to 1000 ml or 50 to 1000 ml relative to 1 mol of the compound represented by formula (4) shown above.

The reaction temperature in the step 1B can be adjusted as appropriate according to the compound represented by formula (3) shown above, R¹—I, the reaction solvent, and the like. The step 1B is preferably carried out, for example, under heating and reflux conditions because the yield of the compound represented by formula (3) shown above can be improved. As a specific example, the reaction temperature can be set as appropriate according to the reaction solvent, and may be set to, for example, 20 to 100° C., and preferably 50 to 100° C., 60 to 90° C., or the like.

In the second manufacturing method of the present invention, the compound represented by formula (3) obtained in the step 1B may be purified. In this case, in the second manufacturing method of the present invention, for example, the reaction product obtained in the step 1B may be subjected to processing such as recrystallization, concentration, neutralization, extraction, and/or drying.

According to the second manufacturing method of the present invention, the yield of the compound represented by formula (3) shown above may be improved.

### Third Manufacturing Method

As described above, a method for manufacturing a compound according to the present invention includes a first step of obtaining a compound represented by formula (3) shown below; and a second step of reacting the compound represented by formula (3) shown below with a compound represented by formula (5) shown below to obtain a compound represented by formula (6) shown below, wherein the first step is carried out using at least one of the first manufacturing method and the second manufacturing method of the present invention described above.

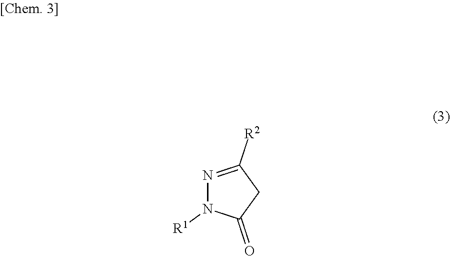

In formula (3) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

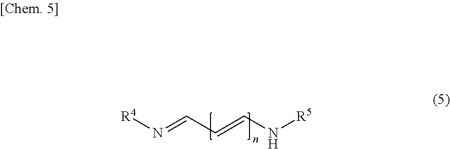

In formula (5) shown above, R⁴ and R⁵ may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

n is an integer of 0 or more.

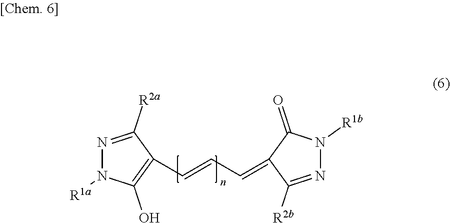

In formula (6) shown above, R^{1a} and R^{1b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

R^{2a} and R^{2b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.

n is an integer of 0 or more.

A feature of the third manufacturing method of the present invention is that the first step of the third manufacturing method is carried out using the first manufacturing method or the second manufacturing method of the present invention described above, and there is no particular limitation on the steps of the third manufacturing method other than the first step and the conditions. In the third manufacturing method of the present invention, the descriptions of the first manufacturing method and the second manufacturing method of the present invention given above can be incorporated by reference.

The compound represented by formula (3), (5), or (6) shown above may be, for example, a salt or an isomer thereof. Examples of the isomer include a tautomer and a stereoisomer. Examples of the tautomer and the stereoisomer include all theoretically possible tautomers and stereoisomers. Also, in the present invention, there is no particular limitation on the stereochemistry of each substituent. In the antioxidant of the present invention, the compound represented by formula (3), (5), or (6) shown above may be, for example, a hydrate or solvate of the compound represented by formula (3), (5), or (6) shown above or a salt thereof.

In the case where the compound represented by formula (3), (5), or (6) shown above has an asymmetric carbon atom, the compound represented by formula (3), (5), or (6) shown above may be present as, for example, a racemate, an R enantiomer or an S enantiomer of the racemate, or a mixture of the R and S enantiomers mixed at any ratio. The compound represented by formula (3), (5), or (6) shown above may have two or more asymmetric centers. In this case, the compound represented by formula (3), (5), or (6) shown above may contain a diastereoisomer and a mixture thereof. In the case where the compound represented by formula (3), (5), or (6) shown above has a double bond in the molecule, the compound represented by formula (3), (5), or (6) shown above may include the form of geometrical isomers such as cis and trans isomers.

Hereinafter, the substituents of the compounds will be described, and examples of each substituent will be given. In the description of each substituent, unless otherwise stated, specific examples of other substituents can be incorporated by reference.

In formula (3) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group or an aryl group, and more preferably represents an ethyl group or a phenyl group.

R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably represents an alkyl group, and more preferably represents an ethyl group.

In formula (5) shown above, R⁴ and R⁵ each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably each represents an alkyl group or an aryl group, and more preferably each represents a phenyl group. R⁴ and R⁵ may be the same or different, but they are preferably the same.

n is an integer of 0 or more, and may be, for example, an integer of 0 to 10, preferably an integer of 0 to 5, 0 to 3, or 1 to 3, and more preferably 0, 1, or 2.

In formula (6) shown above, R^{1a} and R^{1b} each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably each represents an alkyl group or an aryl group, and more preferably each represents an ethyl group or a phenyl group. R^{1a} and R^{1b} may be the same or different, but they are preferably the same. R^{1a} and R^{1b} may be the same as, for example, R¹.

R^{2a} and R^{2b} each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and preferably each represents an alkyl group, and more preferably each represents a methyl group. R^{2a} and R^{2b} may be the same or different, but they are preferably the same. R^{2a} and R^{2b} may be the same as, for example, R².

There is no particular limitation on the combination of R¹, R², R^{1a}, R^{1b}, R^{2a}, R_{2b}, and n, and they can be combined in any way. A specific example of the combination of R¹, R², Ria R^{1b}, R^{2a}, R^{2b}, and n is a combination where R¹ represents an alkyl group or an aryl group, R² represents an alkyl group, R^{1a} and R^{1b} may be the same or different, and each represents an alkyl group or an aryl group, R^{2a} and R^{2b} may be the same or different, and each represents an alkyl group, and n is an integer of 0 to 5. The combination of R¹, R², R^{1a}, R^{1b}, R^{2a}, R^{2b}, and n is preferably a combination where R¹ represents an ethyl group or a phenyl group, R² represents a methyl group, R^{1a} and R^{1b} may be the same or different, and each represents an ethyl group or a phenyl group, R^{2a} and R^{2b} may be the same or different, and each represents a methyl group, and n is 0, 1, or 2. In this case, R⁴ and R⁵ preferably each represents a phenyl group.

Next, the steps of the third manufacturing method of the present invention will be described based on an example. In the third manufacturing method of the present invention, the compound represented by formula (3) shown above is obtained in the first step. The first step can be carried out in the same manner as in the first manufacturing method or the second manufacturing method of the present invention described above.

Next, in the second step, the compound represented by formula (3) shown above is reacted with the compound represented by formula (5) shown above to obtain the compound represented by formula (6) shown above. In the second step, the compound represented by formula (6) shown above can be obtained through, for example, a reduction reaction between two molecules of the compound represented by formula (3) shown above and the compound represented by formula (5) shown above. Specifically, in the second step, for example, a reduction reaction takes place between the amine (—NH—) in the compound represented by formula (5) shown above and the compound represented by formula (3) shown above, and a carbon atom that bonds to the aniline in the compound represented by formula (5) shown above bonds to the carbon atom at the 4 position of the compound represented by formula (3) shown above. Subsequently, in the second step, for example, a reduction reaction takes place between the compound represented by formula (5) shown above and another compound represented by formula (3) shown above, and a carbon atom that bonds to the imine (R⁴—N═C—) in the compound represented by formula (5) shown above bonds to the carbon atom at the 4 position of the compound represented by formula (3) shown above, as a result of which, the compound represented by formula (6) shown above can be obtained.

In the second step, the molar ratio (C3:C5) of the compound represented by formula (3) shown above (C3) and the compound represented by formula (5) shown above (C5) is, for example, 1:0.001 to 1:10, 1:0.01 to 1:5, or 1:0.1 to 1:5.

The second step is preferably carried out in the presence of a base, more specifically, in the presence of a basic substance because the yield of the compound represented by formula (6) shown above can be improved. The description of the base given above can be incorporated by reference.

As the reaction solvent used in the second step, it is possible to use at least one solvent selected from, for example, N,N-dimethylformamide (DMF), acetonitrile, 1,4-dioxane, acetone, ethanol, chloroform, dioxane, xylene, diethyl ether, tetrahydrofuran (THF), dichloromethane (CH₂Cl₂), toluene, and the like. As the reaction solvent, these solvents may be used alone or in a combination of two or more, or a solvent mixture composed of two or more solvents selected from those listed above may be used. In the case where the second step is carried out in the presence of a base, and the base can be used as a reaction solvent, the base may be used as the reaction solvent in the second step. The base that can be used as the reaction solvent may be, for example, an amine such as TEA or DIEA. The reaction solvent is preferably, for example, DMF, acetonitrile, ethanol, an amine, or any of the solvent mixtures thereof because side reactions and the generation of isomers can be suppressed, and the yield of the compound represented by formula (6) shown above can be improved. The amount of the reaction solvent may be, for example, 0.5 to 1000 ml or 50 to 1000 ml relative to 1 mol of the compound represented by formula (3) shown above.

The reaction temperature in the second step can be adjusted as appropriate according to the compound represented by formula (3) shown above, the compound represented by formula (5) shown above, the reaction solvent, and the like. The second step is preferably carried out, for example, under heating conditions because the yield of the compound represented by formula (6) shown above can be improved. As a specific example, the reaction temperature may be, for example, 20 to 100° C., and preferably 50 to 100° C., 60 to 90° C., or the like.

In the third manufacturing method of the present invention, the compound represented by formula (6) obtained in the second step may be purified. In this case, in the second [ [symbol] 3] manufacturing method of the present invention, for example, the reaction product obtained in the second step may be subjected to processing such as recrystallization, concentration, neutralization, extraction, and/or drying.

According to the third manufacturing method of the present invention, the yield of the compound represented by formula (3) shown above can be improved, and thus the yield of the compound represented by formula (6) shown above can be improved.

### EXAMPLES

Next, examples of the present invention will be described. However, the present invention is not limited to the examples given below. Unless otherwise stated, commercially available reagents were used based on the protocols thereof.

### Example 1

It was checked whether the manufacturing methods of the present invention increased the yield.

### (1) First Manufacturing Method

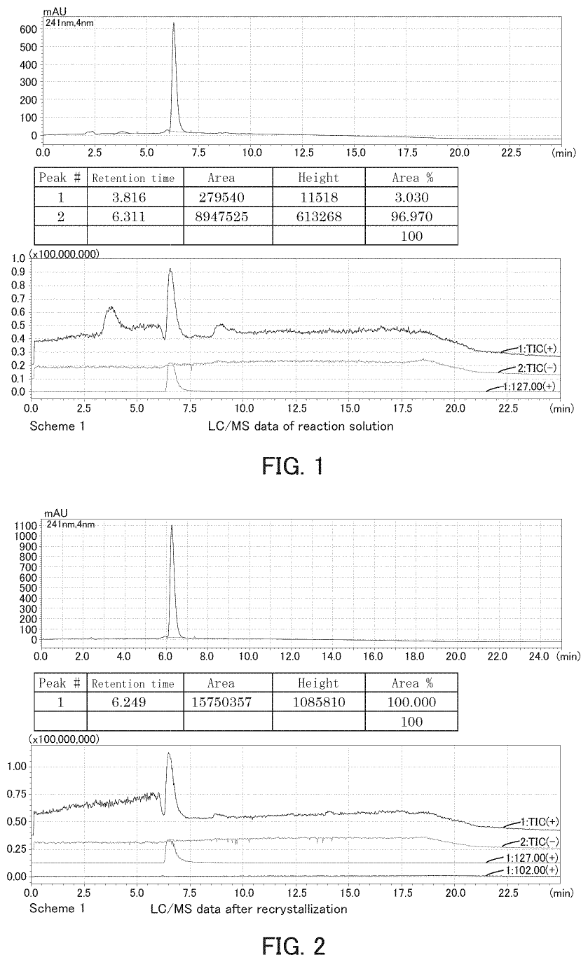

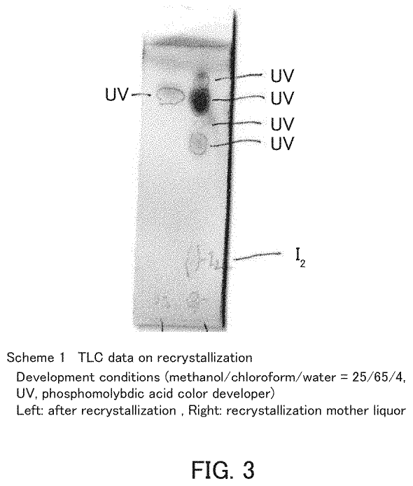

A first manufacturing method according to an example of the present invention was carried out in accordance with a scheme 1 shown below. Specifically, a 50-ml ground-joint test tube was equipped with a stirring bar, a reflux tube, and a three-way cock. Next, in a nitrogen atmosphere, a compound 3 (1.0006 g) corresponding to a compound represented by formula (1) shown above was added to the test tube, and 10 ml of ethanol, triethylamine (1.857 ml, 2 eq), and a compound 4 (843 μl, 1 eq) corresponding to a compound represented by formula (2) shown above were added sequentially. After these had been added, the resulting mixture was stirred at a reflux temperature (room temperature, about 25° C.) overnight (for 18 hours), and then the reaction solution was concentrated to obtain an orange semi-solid product. The obtained semi-solid product was suspended in 20 ml of ethyl acetate, and then filtered and again cleaned with 20 ml of ethyl acetate. Through this, the triethylamine oxalate contained in the semi-solid product was removed. Furthermore, the semi-solid product that had been cleaned was again suspended in an ethyl acetate solution, and about 20 g of silica gel was added and suspended in the ethyl acetate solution, and then the solution was filtered. The obtained residue was eluted using a 10% methanol/ethyl acetate solvent mixture (250 ml). After that, the resulting solution was concentrated using a rotary evaporator to obtain 817.4 mg of a crude product (solid). The obtained solid was dispersed in about 2 ml of acetonitrile, and then the obtained dispersion was subjected to recrystallization (reflux→0° C.) to obtain 658 mg of a light yellow-white solid (with a yield of 78.3%). Mass spectroscopy was performed under the following LC/MS conditions, and it was confirmed that the obtained solid contained 3-methyl-1-ethyl-5-pyrazolone corresponding to a compound represented by formula (3) shown above. LC/MS results of the reaction solution are shown in FIG. 1. LC/MS results of the reaction solution after recrystallization are shown in FIG. 2, and TLC data of the reaction solution before and after recrystallization are shown in FIG. 3.

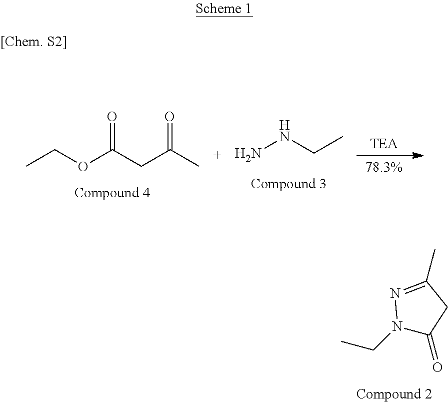

### LC/MS Conditions

Instrument: Shimadzu LCMS-8030
Column: Phenomenex Kinetex 5μ C18, 150 L×2.0 φ(5 μm)
Eluent A: 10 mM NH₄HCO₃+0.10% HCOOH/H₂O
Eluent B: Acetonitrile
Gradient: B conc 0-100 (15 min)
Flow rate: 0.2 ml/min
Column temperature: 40° C.
Detector 1: ESI
Probe voltage: +1.6 kV (ESI-Positive mode),
   −1.6 kV (ESI-Negative mode)
CDL temperature: 250° C.
Block heater temperature: 200° C.
Nebulizing gas flow: 1.5 l/min
Drying gas pressure: 0.02 Mpa
Detector 2: PDA (190 to 800 nm)

### (2) Second Manufacturing Method

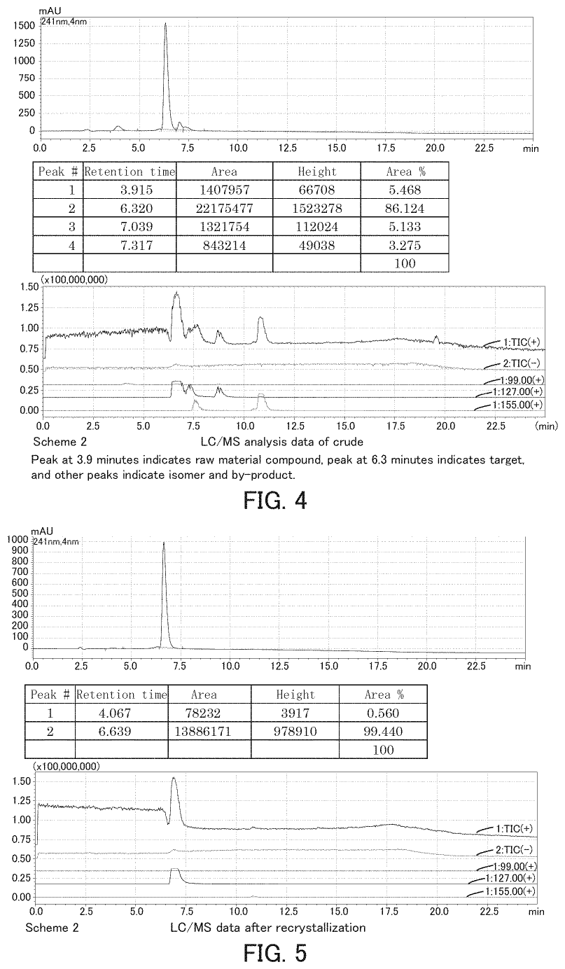

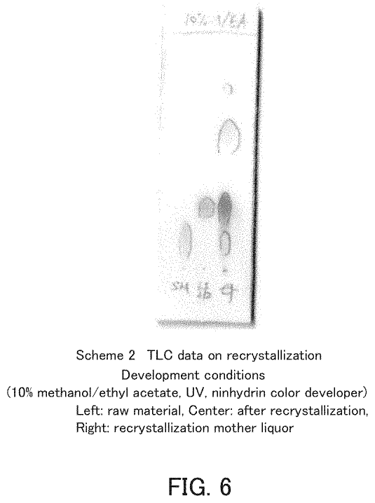

A second manufacturing method according to an example of the present invention was carried out in accordance with a scheme 2 shown below. Specifically, a 50-ml ground-joint test tube was equipped with a stirring bar and a three-way cock. In a nitrogen atmosphere, a compound 1 (a raw material 1, 750.5 mg) corresponding to a compound represented by formula (4) shown above was suspended in 7.5 ml of acetonitrile, EtI (ethyl iodide, 1.849 ml, 3.577 g, 3 eq) was added to the test tube, and the resulting mixture was heated under reflux at 72° C. overnight (for 18 hours). After heating under reflux, the reaction solution was completely dissolved. The obtained reaction solution was concentrated using a rotary evaporator (for removal of excess EtI), and re-dissolved by adding 7.5 ml of water to the obtained light brown syrup. Furthermore, a sodium hydrogen carbonate (NaHCO₃) powder (642 mg, 1 eq) was gradually added to the re-dissolved solution and neutralized. Air bubbles were generated during the neutralization. Also, in order to complete the neutralization, the solution was heated at 100° C. for 10 minutes. After heating, the obtained aqueous solution was cooled, and chloroform was added thereto to extract 3-methyl-1-ethyl-5-pyrazolone corresponding to a compound represented by formula (3) shown above. The extraction process was performed 10 times, where 10 ml of chloroform was added each time. The organic layer recovered in each extraction process was dried using sodium sulfate (Na₂SO₄), and then subjected to filtering and concentration to obtain 578.7 mg of a crude product (light brown solid). The solid was dispersed in about 2 ml of acetonitrile, and subjected to recrystallization (reflux→−20° C.) to obtain 355.8 mg of a white solid with a purity measured under the above LC/MS conditions of 99% or more (with a yield of 36.9%). Mass spectroscopy was performed under the above LC/MS conditions, and it was confirmed that the obtained solid contained 3-methyl-1-ethyl-5-pyrazolone corresponding to a compound represented by formula (3) shown above. LC/MS results of the reaction solution are shown in FIG. 4. LC/MS results of the reaction solution after recrystallization are shown in FIG. 5, and TLC data of the reaction solution before and after recrystallization are shown in FIG. 6. When the method was carried out in the same manner, except that acetonitrile used as the reaction solvent was replaced by DMF, the yield was 37.0%. As a comparative example, when the method was carried out in the same manner, except that EtI was replaced by EtBr (ethidium bromide), the yield was 31.9%.

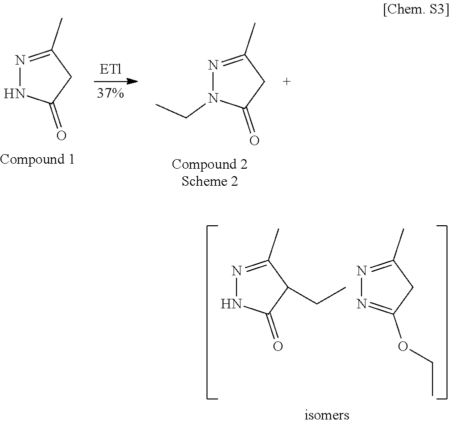

### (3) Third Manufacturing Method

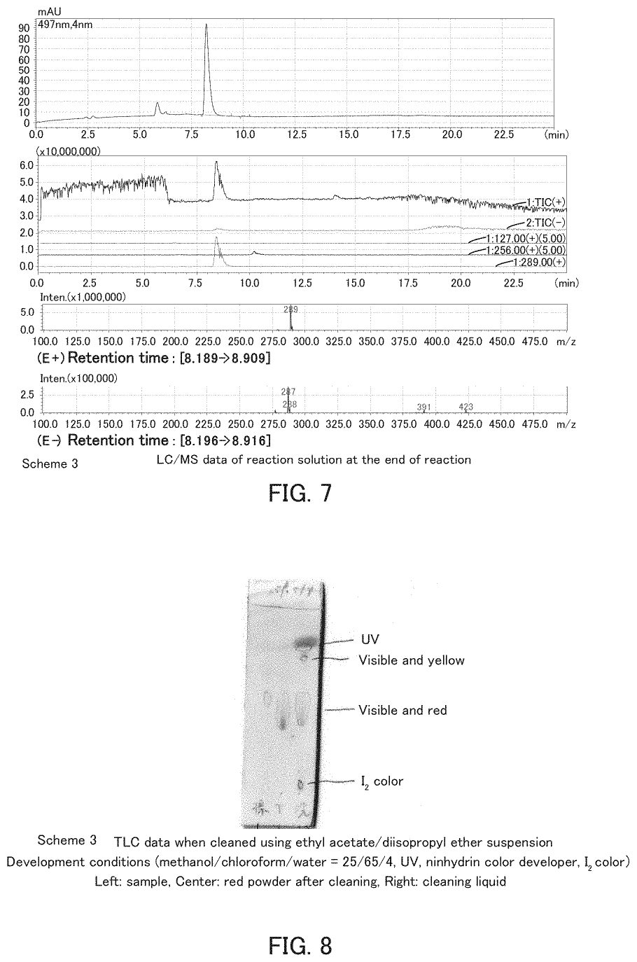

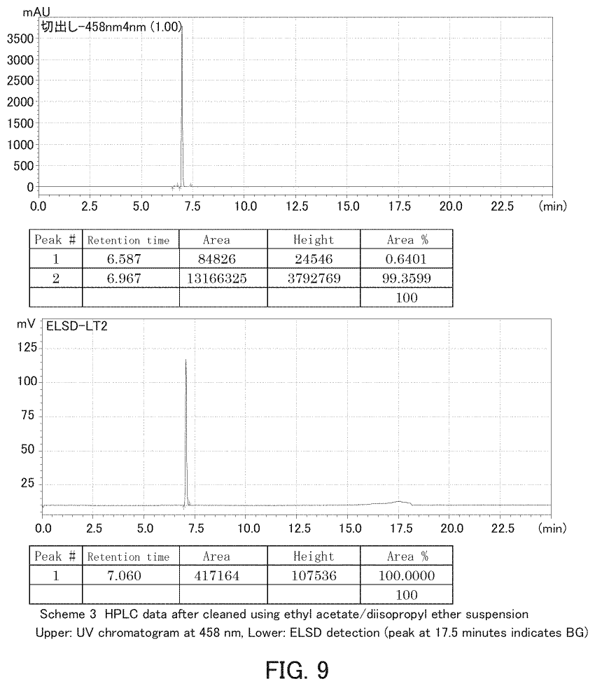

A third manufacturing method according to an example of the present invention was carried out in accordance with a scheme 3 shown below. Specifically, a 50-ml ground-joint test tube was equipped with a stirring bar and a three-way cock. In a nitrogen atmosphere, a compound 2 (154.1 mg, 2.1 eq) corresponding to a compound represented by formula (3) shown above, a compound 5 (149.9 mg, 1.0 eq) corresponding to a compound represented by formula (5) shown above, 1.5 ml of ethanol, and triethylamine (242 μl, 3 eq) were added to the test tube, and the test tube was heated at 78° C. for one hour. The reaction solution was concentrated, and then re-dissolved by adding 10 ml of water and neutralized using 1N—HCl (580 μl, 1 eq). After neutralization, extraction was performed using chloroform (10 ml×4 times, with this operation, the red component was substantially extracted into the organic layer). The organic layer recovered in each extraction process was dried using sodium sulfate (Na₂SO₄), and then subjected to filtering and concentration to obtain 274 mg of a dark red crude product. 5 ml of ethyl acetate was added to the obtained crude product to suspend the crude product. After that, 45 ml of isopropyl ether was added thereto, and the mixture was stirred for a while. After stirring, the mixture was filtered and again cleaned with isopropyl ether. After cleaning, vacuum drying was performed to obtain 147.4 mg of a compound 7 corresponding to a compound represented by formula (6) shown above in the form of a vermillion powder and with an HPLC purity of 99.3% (with a yield of 88.2% relative to that of the compound 5). Mass spectroscopy was performed under the above LC/MS conditions, and it was confirmed that the obtained solid contained 2,4-dihydro-4-[3-(1-ethyl-5-hydroxy-3-methyl-1H-pyrazol-4-yl)-2-propen-1-ylidene]-2-ethyl-5-methyl-3H-pyrazol-3-one (compound 7) corresponding to a compound represented by formula (6) shown above. Also, HPLC was carried out under the following HPLC conditions. LC/MS results of the reaction solution are shown in FIG. 7. TLC results of the dispersion after ethyl acetate and isopropyl ether had been added are shown in FIG. 8, and HPLC data of the dispersion after ethyl acetate and isopropyl ether had been added are shown in FIG. 9. NMR results of the compound 7 in the solid are shown below, from which it was confirmed that the compound 7 was the above-described compound. ¹H-NMR (nuclear magnetic resonance) (600 MHz, DMSO (dimethyl sulfoxide)-d6, AV-600 (available from Bruker Japan K.K.)): δ 1.18 (t, 6H), 2.19 (s, 6H), 3.69 (q, 4H), 7.29 (d, 2H), 8.00 (t, 1H)

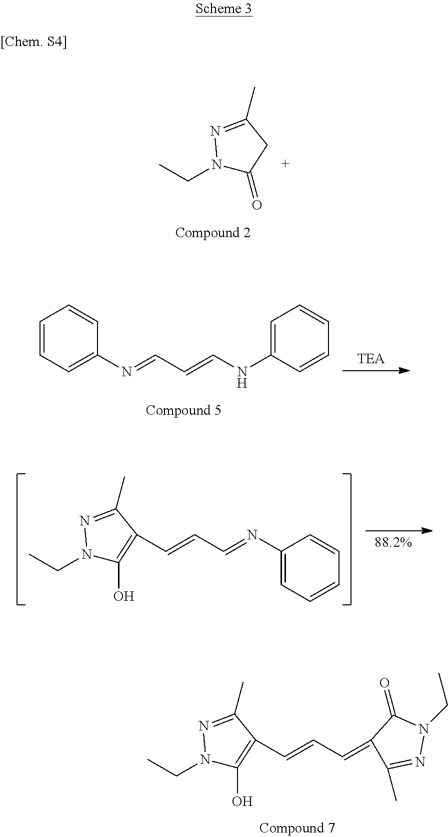

### HPLC Conditions

Instrument: Shimadzu L C-2010A HT/SPD-M20A
Column: Phenomenex Kinetex 5μ C18, 250 L×4.6 φ(5 μm)
Eluent A: 0.1% TFA/H₂O
Eluent B: Acetonitrile
Gradient: B conc 0-100 (5 min)
Flow rate: 1.5 ml/min
Detect PDA (190-800 nm)
   ELSD (Shimadzu ELSD-LT II)
Nebulizer gas pressure: 350 kPa
Temperature: 50° C.
Noise filter: 3 sec.
Gain: 5

From the results above, it was found that, according to the manufacturing methods of the present invention, the compound yield can be improved.

The present invention was described based on the embodiment and the examples given above. However, the present invention is not limited to the embodiment and the examples given above. Various modifications that can be understood by those skilled in the art can be made to the configuration and details of the present invention within the scope of the invention of the present application.

The present application claims priority of Japanese Patent Application No. 2020-128216 filed on Jul. 29, 2020, and the entire disclosure is incorporated herein by reference.

### Additional Statements

A portion or all of the embodiment and the examples given above will be described in the following additional statements. However, the scope of the present invention is not limited thereto.

### Additional Statement 1

A method for manufacturing a compound, the method including a first step of reacting a compound represented by formula (1) shown above with a compound represented by formula (2) shown above to obtain a compound represented by formula (3) shown above.
[In formula (1) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.]
[In formula (2) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R³ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.]

### Additional Statement 2

The method for manufacturing a compound according to additional statement 1, wherein R¹ represents an alkyl group or an aryl group.

### Additional Statement 3

The method for manufacturing a compound according to additional statement 1 or 2, wherein R² represents an alkyl group.

### Additional Statement 4

The method for manufacturing a compound according to any one of additional statements 1 to 3, wherein R³ represents an alkyl group.

### Additional Statement 5

The method for manufacturing a compound according to any one of additional statements 1 to 4,
wherein R¹ represents an alkyl group or an aryl group,
R² represents an alkyl group, and
R³ represents an alkyl group.

### Additional Statement 6

The method for manufacturing a compound according to any one of additional statements 1 to 5, wherein R¹ represents an ethyl group or a phenyl group.

### Additional Statement 7

The method for manufacturing a compound according to any one of additional statements 1 to 6, wherein R² represents a methyl group.

### Additional Statement 8

The method for manufacturing a compound according to any one of additional statements 1 to 7, wherein R³ represents an ethyl group.

### Additional Statement 9

A method for manufacturing a compound, the method including a first step of reacting a compound represented by formula (4) shown above with R¹—I to obtain a compound represented by formula (3) shown above,
wherein R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.
[In formula (4) shown above, R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent.]

### Additional Statement 10

The method for manufacturing a compound according to additional statement 9, wherein R¹ represents an alkyl group or an aryl group.

### Additional Statement 11

The method for manufacturing a compound according to additional statement 9 or 10, wherein R² represents an alkyl group.

### Additional Statement 12

The method for manufacturing a compound according to any one of additional statements 9 to 11,
wherein R¹ represents an alkyl group or an aryl group, and
R² represents an alkyl group.

### Additional Statement 13

The method for manufacturing a compound according to any one of additional statements 9 to 12, wherein R¹ represents an ethyl group or a phenyl group.

### Additional Statement 14

The method for manufacturing a compound according to any one of additional statements 9 to 13, wherein R² represents a methyl group.

### Additional Statement 15

A method for manufacturing a compound, the method including:
a first step of obtaining a compound represented by formula (3) shown above; and
a second step of reacting the compound represented by formula (3) shown above with a compound represented by formula (5) shown above to obtain a compound represented by formula (6) shown above,
wherein the first step is carried out using the method for manufacturing a compound according to any one of additional statements 1 to 14.
[In formula (3) shown above, R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent]
[In formula (5) shown above, R⁴ and R⁵ may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
n is an integer of 0 or more.]
[In formula (6) shown above, R^{1a} and R^{1b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent,
R^{2a} and R^{2b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
n is an integer of 0 or more.]

### Additional Statement 16

The method for manufacturing a compound according to additional statement 15, wherein R¹ represents an alkyl group or an aryl group.

### Additional Statement 17

The method for manufacturing a compound according to additional statement 15 or 16, wherein R² represents an alkyl group.

### Additional Statement 18

The method for manufacturing a compound according to any one of additional statements 15 to 17, wherein R⁴ and R⁵ may be the same or different, and each represents an alkyl group or an aryl group.

### Additional Statement 19

The method for manufacturing a compound according to any one of additional statements 15 to 18,
wherein R¹ represents an alkyl group or an aryl group,
R² represents an alkyl group,
R^{1a} and R^{1b} may be the same or different, and each represents an alkyl group or an aryl group, and
R^{2a} and R^{2b} may be the same or different, and each represents an alkyl group.

### Additional Statement 20

The method for manufacturing a compound according to any one of additional statements 15 to 19, wherein R¹ represents an ethyl group or a phenyl group.

### Additional Statement 21

The method for manufacturing a compound according to any one of additional statements 15 to 20, wherein R² represents a methyl group.

### Additional Statement 22

The method for manufacturing a compound according to any one of additional statements 15 to 21,
wherein R^{1a} and R^{1b} may be the same or different, and each represents an ethyl group or a phenyl group, and
R^{2a} and R^{2b} each represents a methyl group.

### Additional Statement 23

The method for manufacturing a compound according to any one of additional statements 15 to 22, wherein the second step is carried out in the presence of a base.

### INDUSTRIAL APPLICABILITY

As described above, according to the present invention, it is possible to provide a method for manufacturing a compound, with which it is possible to improve the yield of the compound. For this reason, the present invention is preferably applicable to manufacture a predetermined compound. Thus, it can be said that the present invention is very useful in, for example, the field of medicine and the like.

1. A method for manufacturing a compound, the method comprising a first step of reacting a compound represented by formula (1) shown below with a compound represented by formula (2) shown below to obtain a compound represented by formula (3) shown below: 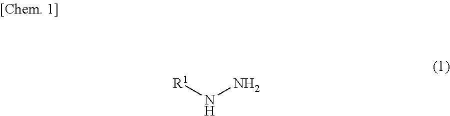
where R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; 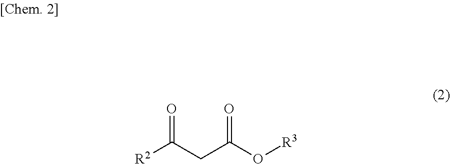
where R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R³ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; and

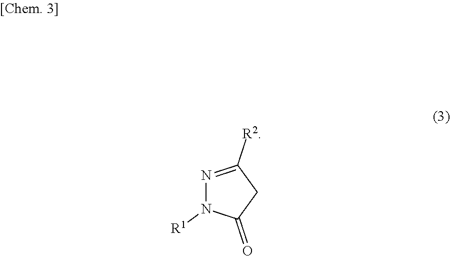

2. The method for manufacturing a compound according to claim 1,
wherein R¹ represents an alkyl group or an aryl group.

3. The method for manufacturing a compound according to claim 1,
wherein R² represents an alkyl group.

4. The method for manufacturing a compound according to claim 1,
wherein R³ represents an alkyl group.

5. The method for manufacturing a compound according to claim 1,
wherein R¹ represents an alkyl group or an aryl group,
R² represents an alkyl group, and
R³ represents an alkyl group.

6. The method for manufacturing a compound according to claim 1,
wherein R¹ represents an ethyl group or a phenyl group.

7. The method for manufacturing a compound according to claim 1,
wherein R² represents a methyl group.

8. The method for manufacturing a compound according to claim 1,
wherein R³ represents an ethyl group.

9. A method for manufacturing a compound, the method comprising a first step of reacting a compound represented by formula (4) shown below with R¹—I to obtain a compound represented by formula (3) shown below,
wherein R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent: 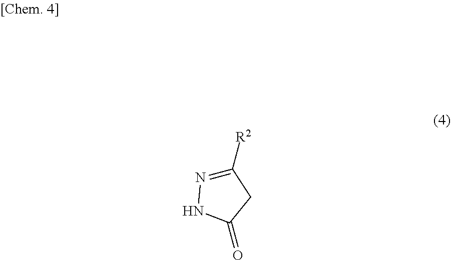
where R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; and

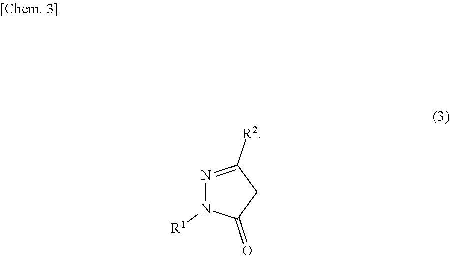

10. The method for manufacturing a compound according to claim 9,
wherein R¹ represents an alkyl group or an aryl group.

11. The method for manufacturing a compound according to claim 9, wherein R² represents an alkyl group.

12. The method for manufacturing a compound according to claim 9,
wherein R¹ represents an alkyl group or an aryl group, and
R² represents an alkyl group.

13. The method for manufacturing a compound according to claim 9,
wherein R¹ represents an ethyl group or a phenyl group.

14. The method for manufacturing a compound according to claim 9,
wherein R² represents a methyl group.

15. A method for manufacturing a compound, the method comprising:
a first step of obtaining a compound represented by formula (3) shown below; and
a second step of reacting the compound represented by formula (3) shown below with a compound represented by formula (5) shown below to obtain a compound represented by formula (6) shown below,
wherein the first step is carried out using the method for manufacturing a compound according to claim 1: 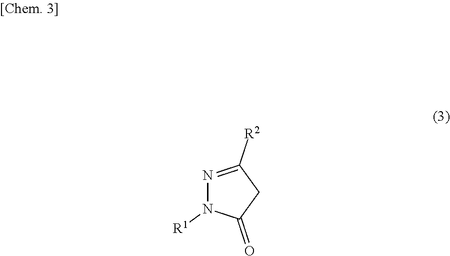
where R¹ represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
R² represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent; 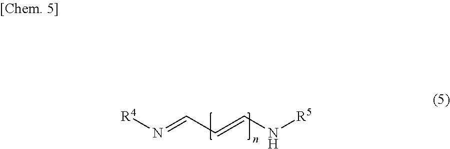
where R⁴ and R⁵ may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
n is an integer of 0 or more; and 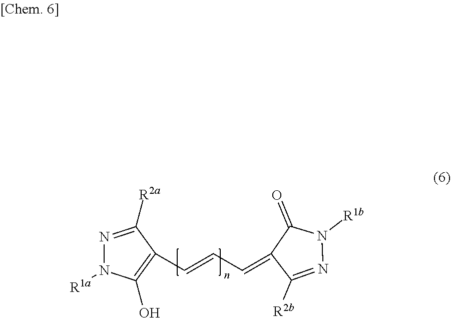
where R^{1a} and R^{1b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent,
R^{2a} and R^{2b} may be the same or different, and each represents a hydrogen atom, a halogen atom, an alkyl group, an amino group, a cyano group, a hydroxy group, a sulfo group, a carboxyl group, an alkoxy group, a hydroxyalkyl group, an acyl group, an alkenyl group, an alkynyl group, or an aryl group optionally having a substituent, and
n is an integer of 0 or more.

16. The method for manufacturing a compound according to claim 15,
wherein R¹ represents an alkyl group or an aryl group.

17. The method for manufacturing a compound according to claim 15,
wherein R² represents an alkyl group.

18. The method for manufacturing a compound according to claim 15,
wherein R⁴ and R⁵ may be the same or different, and each represents an alkyl group or an aryl group.

19. The method for manufacturing a compound according to claim 15,
wherein R¹ represents an alkyl group or an aryl group,
R² represents an alkyl group,
R^{1a} and R^{1b} may be the same or different, and each represents an alkyl group or an aryl group, and
R^{2a} and R^{2b} may be the same or different, and each represents an alkyl group.

20. The method for manufacturing a compound according to claim 15,
wherein R¹ represents an ethyl group or a phenyl group.

21. The method for manufacturing a compound according to claim 15,
wherein R² represents a methyl group.

22. The method for manufacturing a compound according to claim 15,
wherein R^{1a} and R^{1b} may be the same or different, and each represents an ethyl group or a phenyl group, and
R^{2a} and R^{2b} each represents a methyl group.

23. The method for manufacturing a compound according to claim 15,
wherein the second step is carried out in the presence of a base.
